(19) United States
(12) Patent Application Publication
(10) Pub. No.: US 2025/0037880 A1
(43) Pub. Date: Jan. 30, 2025

(54) ARTIFICIAL-INTELLIGENCE TECHNIQUES FOR FORECASTING INTENSITY OF UNINTENDED MOTOR MOVEMENTS

(71) Applicant: RUNE LABS, INC., San Francisco, CA (US)

(72) Inventors: Brian Marc Pepin, San Francisco, CA (US); Aiden Arnold, Victoria BC (CA); Ram Balasubramanian, Berkeley, CA (US)

(73) Assignee: RUNE LABS, INC., San Francisco, CA (US)

(21) Appl. No.: 18/786,328

(22) Filed: Jul. 26, 2024

Related U.S. Application Data
(60) Provisional application No. 63/516,397, filed on Jul. 28, 2023.

(51) Int. Cl. G16H 50/50; G16H 50/30

(52) U.S. Cl. CPC G16H 50/50; G16H 50/30

(57) ABSTRACT

The present disclosure relates to a method and system for acquiring and analyzing multi-modal data to monitor, forecast, and manage one or more symptoms of the neurodegenerative disorders such as Parkinson disease of a subject. The multi-modal data may include sensor data from a wearable sensing device, medications data, symptom-intensity scores for one or more symptoms, and mobility metrics of the subject. A predicted symptom-intensity score (e.g., absolute or relative value) may be generated for each of the one or more symptoms using a symptom-forecasting model (e.g., a machine learning model) and for each of one or more future time periods. Based on the predicted symptom-intensity scores, a trend can be generated for a selected time period. The disclosed system may output a result that comprises of intervention actions, such as medication administration, physical activity, or any other intervention that can be used to control symptoms severity.

### CROSS-REFERENCE TO RELATED APPLICATIONS

This application claims the priority to and the benefit of U.S. Provisional Application No. 63/516,397, filed on Jul. 28, 2023, entitled “Artificial-Intelligence Techniques for Forecasting Intensity of Unintended Motor Movements”, which is hereby incorporated by reference in its entirety for all purposes.

### BACKGROUND

Neurodegenerative disorders or degenerative nerve disorders are linked to the health and functioning of different nerve cells and/or neurons including sensory nerve cells, motor nerve cells, and interneurons. Such disorders affect several activities including movement, balance, speaking, breathing, and various other stimuli responses of a subject. The neurodegenerative disorders indicate an incoordination between the central nervous system (CNS) and various other organs and body parts of the subject. For example, the subject may experience difficulty in controlling a limb, tremors, jerky movements and spasms (dystonia), slurred speech, problems with balance and co-ordination etc., due to one or more neurodegenerative disorders.

Parkinson's disease (PD) is a progressive neurodegenerative disorder affecting the nervous system and the parts of the body controlled by the nervous system. A person diagnosed with Parkinson's disease may experience tremors, muscle stiffness, slowness of movement, or any other unintended movements due to involuntary muscle actions (e.g., dyskinesia). The symptoms of Parkinson's disease gradually intensify or get worse as the disease advances over time. Treatments and corrective therapies can reduce the effect of the symptoms, relieve pain, and increase mobility of the subject.

Moreover, a person may experience an “off state” in which the effect of symptoms returns as the effect of a medication or corrective therapy reduces. Furthermore, a person may experience a delayed “on state” that pertains to delayed effect of the medication or corrective therapy. Levodopa (L-Dopa) treatment is advised to increase concentration of dopamine in the subject with Parkinson's disease. Additionally, treatment measures (e.g., adjusting the dosage of L-Dopa, taking Duodopa to stabilize the amount of dopamine, and other corrective techniques including deep brain stimulation) can further reduce the effect of the symptoms thereby improving quality of life of the subject.

Thus, there is a need for improved techniques, methods, and systems to monitor the effect of symptoms associated with the neurodegenerative disorders like Parkinson's disease, dyskinesia, dystonia, and the like, in the subject. More specifically, an accurate and improved monitoring of the effect of symptoms associated with the neurodegenerative disorders may aid in planning an effective treatment plan for the subject, medication of the subject and dosage associated with the treatment.

Further, there is a need for improved techniques to assess the severity of the symptoms over a period of time by predicting a trend and/or pattern associated with the symptoms. The need to predict the trend and/or pattern associated with symptoms of the neurodegenerative disorders aims to predict a future occurrence of one or more symptoms and to recommend corrective measures to decrease or stabilize the effect of the symptoms of the subject.

### SUMMARY

Some embodiments of the present disclosure relate to use of sensor data from a wearable sensing device to monitor, forecast and manage one or more symptoms of neurodegenerative disorders of a subject. A computer-implemented method includes obtaining the sensor data of the subject for one or more past or current time periods. The time period can be predefined or can be selected by a user or the subject through an interface, for example, 1 min, 5 min, 10 min, 20 min and the like. The sensor data may comprise of data collected by an accelerometer or a gyroscope of the wearable sensing device. The wearable sensing device can be a user device, for example, smartwatch, smartphone etc. or a dedicated device with sensors and a transceiver.

In some embodiments, one or more features may be extracted based on the sensor data for each time period of the one or more past or current time periods. In some instances, a symptom-intensity score may be determined for each time period of the one or more past or current time periods based on at least a portion of the sensor data that corresponds to the time period. The one or more features that are extracted for the time period may be determined using the symptom-intensity score.

A predicted symptom-intensity score for one or more symptoms of the subject may be generated based on the extracted one or more features in a specific time frame using a symptom-forecasting model. The specific time frame may include one or more time periods, for example, last 30 min, 4 hours, 12 hours, 2 days, 1 week and the like. The predicted symptom-intensity score may represent a predicted intensity of the one or more symptoms during a future time period. The predicted symptom-intensity score corresponds to the predicted intensity of the one or more symptoms of Parkinson's disease. Similarly, the predicted symptom-intensity score may correspond to a predicted intensity of tremors or a predicted intensity of dyskinesia. In some instances, the predicted symptom-intensity score may be generated using a trend. The trend can be generated using the symptom-intensity scores corresponding to the one or more past or current time periods. Moreover, the predicted symptom-intensity score is based on the trend.

In some embodiments, the predicted symptom-intensity score may further be generated based on medical information comprising dosage information associated with a treatment of the subject, medication time delta information, and information regarding effect of the treatment on the subject. The medical information or medications data may be retrieved from an electronic health record or can be provided by the subject through an interface. In some other embodiments, the predicted symptom-intensity score may be generated based on multi-modal data comprising the sensor data, the medical information, and mobility metrics of the subject. The mobility metrics of the subject can be derived from the sensor data.

According to the disclosed technique, a result or output may be generated based on the predicted symptom-intensity score. The result may provide a basis for a recommendation or includes the recommendation to perform an intervening action to avoid symptoms in accordance with the predicted symptom-intensity score. The result or output may include an alert that the one or more symptoms of the neurodegenerative disorder may get worse in the future time period without the intervening action. The result, the trend, and/or the alert may be presented on a device (e.g., smartphone, tablet, laptop etc.) of the subject, caregiver, or a clinician or may be transmitted to another device, remote device, or a cloud server.

In some embodiments, a system is provided that includes one or more data processors and a non-transitory computer readable storage medium containing instructions which, when executed on the one or more data processors, cause the one or more data processors to perform part or all of one or more methods disclosed herein.

In some embodiments, a computer-program product is provided that is tangibly embodied in a non-transitory machine-readable storage medium and that includes instructions configured to cause one or more data processors to perform part or all of one or more methods disclosed herein.

In some embodiments, a system is provided that includes one or more means to perform part or all of one or more methods or processes disclosed herein.

The terms and expressions which have been employed are used as terms of description and not of limitation, and there is no intention in the use of such terms and expressions of excluding any equivalents of the features shown and described or portions thereof, but it is recognized that various modifications are possible within the scope of the invention claimed. Thus, it should be understood that although the present invention as claimed has been specifically disclosed by embodiments and optional features, modification and variation of the concepts herein disclosed may be resorted to by those skilled in the art, and that such modifications and variations are considered to be within the scope of this invention as defined by the appended claims.

### BRIEF DESCRIPTION OF THE DRAWINGS

Various embodiments are described hereinafter with reference to the figures. It should be noted that the figures are not drawn to scale and that the elements of similar structures or functions are represented by like reference numerals throughout the figures. It should also be noted that the figures are only intended to facilitate the description of the embodiments. They are not intended as an exhaustive description of the disclosure or as a limitation on the scope of the disclosure.

FIG. 1 shows an example network for performing a method of symptom monitoring, symptom forecasting, and symptom management of a subject affected with one or more neurodegenerative disorders.

FIG. 2 shows an illustrative example of multi-modal data acquisition for symptom-forecasting and management of the subject in accordance with some embodiments of the present disclosure.

FIG. 3 illustrates an exemplary pipeline to process buffered data or the multi-modal data for a symptom-forecasting model that predicts one or more symptom-intensity scores of the subject in accordance with some embodiments of the present disclosure.

FIG. 4 shows an illustrative example of a first interface of a user interface of an application that can run on a computing device in accordance with some embodiments of the present disclosure.

FIG. 5 shows an illustrative example of a second interface of the user interface of the application that can run on the computing device in accordance with some embodiments of the present disclosure.

FIG. 6 shows an example flowchart of a system to predict the one or more symptom-intensity scores of the subject in accordance with some embodiments of the present disclosure.

FIG. 7 shows an example illustration of a computer system in which various embodiments of the present disclosure may be implemented.

### DETAILED DESCRIPTION

Some embodiments of the present disclosure relate to an improved data-driven method and system for symptom forecasting and symptom management of a subject affected with one or more neurodegenerative disorders. Sensor data is obtained, which may include a time-series signal corresponding to movement of the subject, a physiological measurement, etc. The sensor data may include data collected by an accelerometer, gyroscope, light sensor, etc. For each time period of a set of time periods, a symptom-intensity score can be generated by processing the sensor data from one or more sensors corresponding to the time period using a symptom-intensity model (or a symptom-scoring model). A symptom-intensity score may identify whether a given symptom (e.g., a tremor, dyskinesia) was detected during the time period, a cumulative period of time during the time period for which it is estimated that the symptom was occurring, what percentage of the time period it is estimated that the symptom was occurring, an estimated intensity of the symptom during the time period (e.g., or a maximum, average, median, or other statistic pertaining to multiple symptoms—and/or lack thereof—occurring throughout the time period), etc. In some instances, a symptom-intensity score is generated based on an estimated occurrence of, severity of, and/or frequency of multiple frequencies, which may be determined based on measurements from multiple sensors.

The intensity of the symptoms gradually intensify as the neurodegenerative disorder advances over time. For instance, dyskinesia can become more pronounced and/or more common as Parkinson's disease progresses. That said, when and the extent to which a symptom occurs may depend on many factors, such as in which activities the subject is engaging currently or in a recent time period (e.g., exercising, gardening, shopping, siting), a time of day, and/or which medications and/or corrective therapies have been taken (e.g., in recent or longer term time periods) by the subject. Thus, it may be difficult to predict symptoms for specific individuals and/or to predict when to administer a medication to a specific individual to stave off undesirable future symptoms (e.g., in terms of reducing the severity, timing or occurrence of the symptoms, etc.). Currently, many subjects are provided with a set schedule indicating when to take medication. The schedule is determined based on the type and amount of medication that has been prescribed. However, the set schedule usually assumes that the subject is in the same physical state each day of the week, which is not the case in real-life. According to some embodiments, a technical solution is provided in the present disclosure to a technical problem of monitoring and predicting symptoms intensity of the neurodegenerative disorder in an accurate and quick manner for timely interventions.

In some embodiments, for each time period of the set of time periods, the symptom-intensity scores (e.g., “tremor %”, “dyskinesia %”) can be aggregated with data corresponding to one or more other variables to form an input data set. The one or more other variables may include (for example) one or more mobility metrics, medication information (e.g., identifying-relative to a current time period-when a subject did take a medication, reported taking a medication and/or was supposed to take a medication and potentially a corresponding dosage), and/or a time of day associated with the time period. For example, a mobility metric may estimate a number of steps taken or a distance walked/run etc., a statistic (e.g., maximum, average, median) of an estimated speed that a subject moved (e.g., across the entire time period or when the subject was estimated to be walking/running), an estimated stride length, an estimated double support percentage (indicating a percentage of time for which it is estimated that both of the subject's feet are on the ground), etc. Thus, it can be appreciated that the input data set can be a multi-modal data set.

Due to the multi-modal data set including different types of variables, the corresponding data points may be collected at different frequencies. As the data points are collected, the data points can be stored in a time window buffer also referred hereinafter as a buffer, thereby accumulating the data points in the buffer and the length of the buffer. The buffer can be of a defined time period (e.g., equal to or less than: 10 minutes, 20 minutes, 30 minutes, 45 minutes, 60 minutes, etc.).

At a featurization stage, a feature may be generated for the time period based on the multi-modal data stored in the buffer. Each feature may correspond to data of one or more modalities. For example, a feature may include or relate to a statistic (e.g., average) pertaining to the one or more symptom-intensity scores corresponding to multiple prior time periods. Features extracted for a time period are stored in a feature-lag buffer that also stores previously extracted feature values corresponding to past time periods or past data of the buffer.

In some instances, once the feature lag buffer is full, it is combined with features from the current time period and fed to the symptom-forecasting model (e.g., a machine learning model), which can generate a result that predicts a symptom intensity at one or more future time periods or subsequent time periods. The result may further predict a trend in symptom intensity, whether a symptom intensity will increase, decrease, or remain stable (e.g., within a given future time period), etc. The one or more subsequent time periods may (collectively) extend to, for example, at least 10, at least 20, at least 30, at least 60, at least 90, or at least 120 minutes in the future. In some instances, a relative or absolute symptom intensity may be predicted for each of multiple future time periods. For example, a symptom intensity for the future time periods such as half-hour intervals or one-hour intervals in a remainder of a day, predefined larger time period (e.g., 24 hours), etc. can be estimated.

One or more of the predicted symptom-intensity scores can be presented to the subject via an interface on a device, such as a smart watch or a smart phone. The device may be the same or different than one that includes the sensors used to collect the sensor data. The interface can be configured with one or more input components configured to receive user input (user feedback and user preferences e.g., time period) that specifies details about how to update the prediction of the symptom-forecasting model. For example, the one or more input components can be configured to receive input that identifies: (i) sensitivity of symptom changes (e.g., +/−5 minutes, +/−10 minutes) used in the decision process to identify a trend, (ii) the prediction time window (e.g. 10, 30 or 60 minutes), and/or (iii) the symptom being predicted (e.g. tremor, dyskinesia).

Additionally or alternatively, an alert criterion can be evaluated using at least one of the predicted symptom-intensity scores to determine whether to present an alert that may indicate that it is predicted that the intensity, probability, or frequency of symptoms will worsen without an intervening action (e.g., taking a medication, exercising, etc.). The alert may identify one or more suggested intervening actions and/or may recommend an intervening action (e.g., taking a medication, exercising, etc.). The alert criterion may be configured to be satisfied (for example) when it is predicted that a symptom intensity will increase (e.g., generally or by at least a threshold degree) or when a request from a user (e.g., the subject) has been received within a defined period of time for the alert.

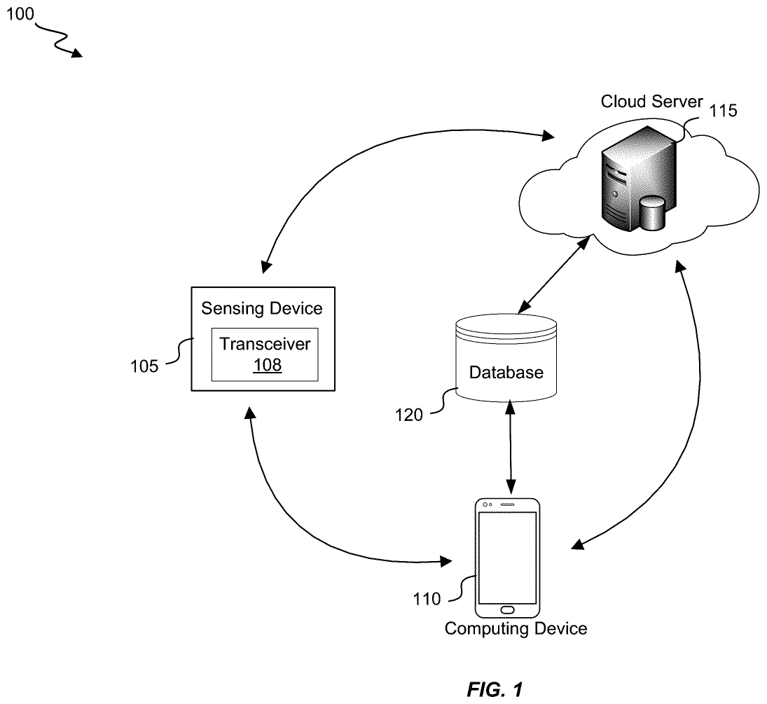

FIG. 1 shows an example network for performing a method of symptom monitoring, symptom forecasting, and symptom management of a subject affected with one or more neurodegenerative disorders. Exemplary network 100 comprises a sensing device 105, a computing device 110, a cloud server 115, and a database 120. The sensing device 105 can be a wearable user device such as smartwatch, or a standalone device of small form factor that can capture, record, process, and transmit sensor data (e.g., shimmer® sensing module). In some instances, the sensing device 105 can be a user device, for example, smartphone attached to the subject body. In some cases, the sensing device 105 and the computing device 110 both can be same such as the smartphone of the subject.

The sensing device 105 can include one or more sensors that may collect signals corresponding to biological, physiological and/or behavioural information (e.g., motor activity) of the subject. The signals may be acquired or collected in periodic intervals or continuously while the subject may perform normal activities of daily life. The one or more sensors may include but not limited to accelerometers, gyroscopes, proximity sensor, light sensor, electromyography (EMG) sensor, photoplethysmography (PPG) sensor. For example, the sensing device 105 may include an accelerometer and/or gyroscope that can be used to collect signals that can be processed to estimate one or more types of movement (e.g., a step count, movement intensity, tremor strength, movement jerkiness, dyskinesia severity, etc.). The sensing device 105 may be comprised of hardware and software components (e.g., firmware, signal acquisition and processing code, or operating system) and can be used to perform initial processing. The initial processing may include amplification of signals recorded by the one or more sensors, determining a differential signal (e.g., in case of surface EMG), applying a filter (e.g., to remove signals around 50-60 Hz or to focus on frequency bands of interest), and/or down sampling the signals.

The sensing device 105 may include a transceiver 108 to communicate with the computing device 110 and/or the cloud server 115. The transceiver 108 can be configured to communicate sensor data recorded by the sensing device 105 to the computing device 110 in real-time. In some instances, the transceiver 108 and/or the sensing device 105 can be configured to receive an instruction or request from the computing device 110, such as an instruction to send the biological, physiological and/or behavioural information of the subject corresponding to the “ON” state and “OFF” state pertaining to the effect of medication on the subject. In some instances, the transceiver 108 is further configured to receive a request to send the information and/or data points to the computing device 110 corresponding to a particular time duration not limited to the “ON” state and “OFF” state. Moreover, the communication between the sensing device 105 and the computing device 110 can occur using any of a variety of commercially available protocols, such as a wireless network, including a short-range connection (e.g., a Bluetooth, Bluetooth low energy (BTLE), or ultra-wideband connection) or over a Wi-Fi network, such as the Internet, etc.

In some other embodiments, the sensing device 105 may be configured to record and transmit wirelessly the sensor data in encrypted format to the cloud server 115 or the computing device 110 for further display, storing, processing, and analysis. The computing device 110 may be a device operated by the subject, a caretaker of the subject, and/or an entity facilitating medical monitoring or treatment for the subject. The computing device 110 may include a mobile device (e.g., a smart phone, smartwatch), personal digital/data assistants (PDA), a tablet, a laptop, a desktop computer, a computer server, and the like.

The techniques disclosed in the present disclosure for symptom monitoring, forecasting and management can be implemented in an application, for example, a mobile-app that may be run on the computing device 110 or a cloud-app to run on the cloud server 115. For instance, the application may process and analyze the acquired sensor data and can display the results (e.g., intensity score values of individual symptoms for a selected time frame, trends, intervening actions etc.) for the subject or the caregiver on the computing device 110. The application on the computing device 110 (or the user device) may transmit or upload the results to the cloud server 115. The computing device 110 may store the raw sensor data, processed sensor data, intensity scores, mobility metrics, or other metrics of the subject associated with each time period into a local database and/or in the database 120. In some instances, metadata may also be stored in the local database or in the database 120. The metadata may include subject identity, type of sensor data collected, sensor placement location (e.g., upper arm), time and date etc.

According to some embodiments, the database 120 may include one or more electronic health record (EHR) databases and can be accessed via the computing device 110 or the cloud server 115. The database 120 may include demographics of the subject, medical history, clinical notes, medications (e.g., prescribed medications, dosages, frequencies, and prescription history etc.). The database 120 may further include historical data corresponding to past time periods of other subjects such as sensor data, medication data, behavioral data, disease history, one or more symptoms score and the like. In some instances, the subject, a caregiver of the subject, or an entity facilitating medical monitoring or treatment for the subject can also provide or update the medical information using the application such as a medical condition of the subject. The information provided by the subject, or a user of the application may include but is not limited to medication, recently taken dosages (e.g., last medication taken), physical activities performed (e.g., walk, run, resting in last few hours or past time periods), frequently occurring symptoms, side-effects of the medication and the like.

In some instances, the computing device 110 may process the information and/or data points collected by the wearable sensors of the sensing device 105 may further be analyzed in accordance with the medical information provided by the user or may be obtained from the database 120. Examples of functions performed by the computing device 110 or by the user device may include but are not limited to obtaining (e.g., from a user device, a sensor, or a local memory) sensor data associated with the subject; generating one or more symptom-intensity scores (also referred herein as symptom-severity scores) based on the sensor data and using a symptom-intensity model; extracting one or more features from the symptom-intensity scores and/or sensor data for a specific time frame, generating a predicted symptom-intensity score for each of the one or more symptoms of the subject and for a future time period; generating one or more predicted symptom-intensity scores by processing at least the symptom-intensity scores in the specific time frame using a symptom-forecasting model; and outputting a result that is based on and/or includes the one or more predicted symptom-intensity scores to the application (e.g., mobile-app on the computing device 110, or the cloud-app on the cloud server 115) or to another computing device, and the like.

In some instances, the symptom-forecasting model may be implemented in the cloud server 115 as a cloud service or the cloud-app. The cloud server 115 may receive, process, analyze the sensor and other data (e.g., medication history, physical activities) and can output predicted symptom-intensity scores, trend, intervening actions etc. The cloud server 115 or the cloud service may transmit the results (e.g., scores, alerts, intervening actions etc.) to the computing device 110 (e.g., device of the subject and/or the caregiver). In some other instances, the symptom-forecasting model may be implemented in the computing device 110 and the results can be stored in the local database of the computing device 110 and/or in the database 120. The caregiver or the medical provider associated with treating the subject can access the results in the database 120, for example, one or more symptom intensity scores corresponding to current and past time periods, using the cloud service or the cloud-app running on the cloud server.

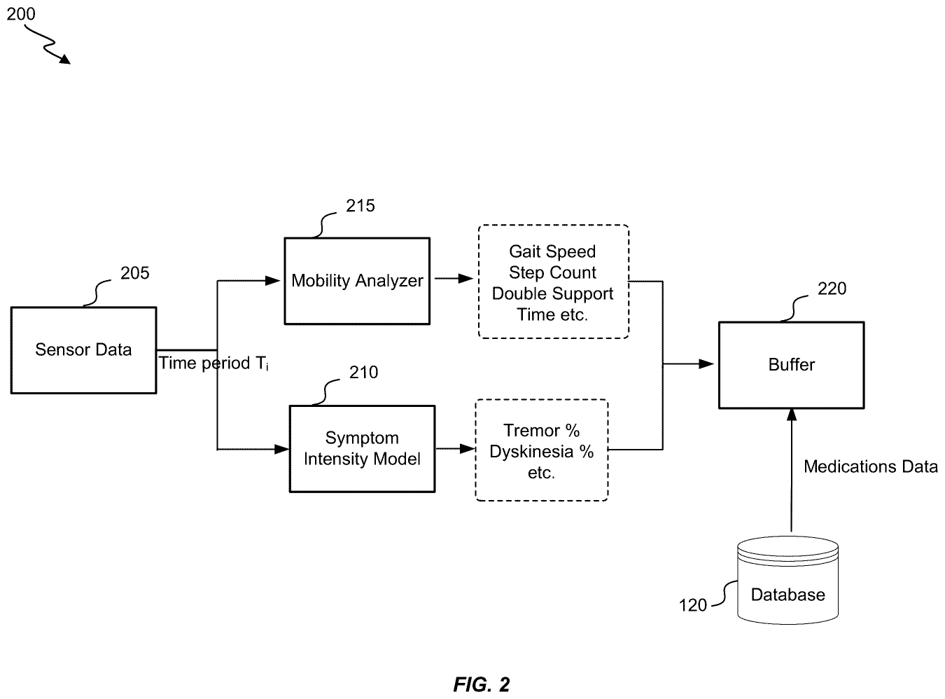

FIG. 2 shows an illustrative example of multi-modal data acquisition for symptom forecasting and management of the subject in accordance with some embodiments of the present disclosure. The sensor data 205 acquired from the sensing device 105 may be processed further at the computing device 110 to generate mobility metrics by a mobility analyzer 215 and the one or more symptom intensity scores using a symptom-intensity model 210. For example, the symptom-intensity model 210 and the mobility analyzer 215 may estimate symptom score (e.g., tremor % or score or intensity, dyskinesia % etc.) and physical activity metrics (e.g., gait speed, steps count etc.) respectively, based on the sensor data 205 of the subject corresponding to a time period Ti. In some instances, some or all of the computations performed by the computing device 110 may be implemented using the cloud server 115.

The symptom-intensity model 210 may generate a symptom-intensity score for each time period during which the sensor data 205 was collected. In some instances, the symptom-intensity model 210 may be configured to receive data corresponding to one or more modalities and/or one or more sensors. The symptom-intensity model 210 may be a rule-based model or a machine-learning model, such as a regression model, neural network, or clustering model. For example, the symptom-intensity model 210 may transform time-series data (or the sensor data 205) into frequency-space data and may identify power at each of one or more frequency bands. In some instances, a weight is assigned to each of the one or more frequency bands (e.g., based on a clustering algorithm, principal component analysis, independent component analysis, neural network, etc.), and the symptom-intensity score is calculated by convolving the weights of various bands with the time-period-associated power values of the bands.

In some embodiments of the present disclosure, the sensor data 205 may include accelerometer signals, gyroscope signals, or EMG signals. These signals provide objective measures that can be leveraged with regression techniques to estimate the intensity of symptoms caused by neurodegenerative disorders (e.g., Parkinson's disease). EMG signals can detect muscle activity and may provide insights into the amplitude and frequency characteristics of unintended motor movements such as tremors, dyskinesia, or dystonia. Accelerometers and gyroscopes provide information on movement patterns and orientation changes and can be used to quantify tremor severity and related motor impairments. For example, accelerometer data corresponding to each time period may be transformed into time-series displacement data, which may be transformed into the frequency space. A power within a given frequency band may be used as a proxy for (or otherwise used to calculate) tremor strength or dyskinesia severity. For example, power at a low-frequency band (e.g., 1 Hz) may be indicative of dyskinesia. As another example, an estimate of tremor severity can be generated based on the power within one or more of a 3-7 Hz band, a 4-9 Hz band, and a 7-12 Hz band.

Regression techniques, such as linear regression or more sophisticated machine learning algorithms like support vector regression or neural networks, can utilize these signals (e.g., accelerometer signals, gyroscope signals, or EMG signals) as input features to predict symptom intensity scores. In some instances, the symptom-intensity model 210 can be based on regression techniques. The model may be trained on a labeled dataset that may include signals or signal features along with clinically assessed symptom severity. The labeled dataset may be comprised of the sensor data 205 of one or more subjects acquired during plurality of past time periods and corresponding symptom-intensity scores (e.g., provided or verified by the clinician).

In some instances, the mobility analyzer 215 may process the sensor data 205 (e.g., acceleration and gyroscope signals) using signal processing techniques and/or machine learning algorithms to generate mobility metrics. The mobility metrics may include but not limited to gait parameters such as gait speed, stride length, step height, cadence, single support time, double support time, stance time, swing time, variability in gait parameters, and symmetry between left and right limbs. The mobility metrics may act as objective indicators of mobility impairments in the subjects with neurodegenerative disorders such as Parkinson disease. These metrics can be used to quantify and monitor changes in movement patterns over time and can facilitate in tracking of disease progression and to evaluate the effectiveness of therapeutic interventions in improving functional outcomes.

In some other instances, the one or more symptom-intensity scores may be analyzed in view of other data (e.g., corresponding to one or more other types of variables). For example, the one or more symptom-intensity scores that indicates (or can be processed to indicate) a severity of tremor or of dyskinesia for the subject can be processed in combination with other data points that may represent a different motor characteristic, physiological metric, biometric data point, medication information, and the like. Such processing may include generating a single data set that includes the one or more symptom-intensity scores and the other data points and processing the single data set; generating an interim result based on the sensor data and processing the interim result and the other data points to generate a result; generating an interim result based on the other data points and processing the interim result and the sensor data to generate a result; etc. For example, the medication information may identify one or more times and/or one or more dosages that a subject received (or reported receiving) of a particular medication. Such medication information may be obtained from the database 120 (e.g., which may store a schedule of times at which the subject is supposed to or had reported taking a particular medication dosage). Similarly, the medication information may further be received separately via input from or a communication from the subject or the caregiver of the subject using the application (e.g., the mobile-app or the cloud app).

In some instances, the multi-modal data including the one or more symptom-intensity scores, underlying sensor data and/or other data may be sampled at different frequencies. For example, a symptom-severity score (or symptom-intensity score) may be generated for every 10-minute interval, whereas another medication-intake information may be observed every 30 minutes. In some instances, a buffer 220 can be configured to store the samples of the information and/or data points corresponding to the desired sampling rate. In some instances, the buffer 220 may be a temporary or a permanent storage location associated with the intelligent symptom monitoring, forecasting, and management system of the present disclosure. The buffer 220 may include but is not limited to data storage devices such as memory of the computing device 105, memory of the cloud server 115, memory of cloud computing resources, and the like. In some instances, the buffer may store signals or multi-modal data corresponding to multiple time periods. In some embodiments, the sensor data 205 may be streamed to the buffer 220 directly, and then the sensor data 205 associated with each time period can be accessed from the buffer 220 to extract features including mobility metrics, symptom intensity scores etc.

As an exemplary scenario, a subject Y may start experiencing involuntary muscle actions, such as shaking (say about 0-5% tremors in the morning) gradually progressing to let's say 40%, associated with the degree of tremors. As the day progresses further, the degree of tremors fluctuates between 40%-65% till 1300 hours and gradually comes down to 0-5% by 1700 hours. In this case, the obtained information and/or data points may be sampled for example, for every 30 minutes of the day. The sampling rate (e.g., sampling of the information and/or data points at every 30 minutes) may be associated with a capacity of the buffer 220 associated with devices of the intelligent symptom monitoring, forecasting, and management system. Additionally, a sample comprising the obtained information and/or data points may further be sampled (e.g., for every 5, 10, or 15 mins of the day) to further assess the degree and/or severity of the symptoms experienced by the subject (person Y in this case).

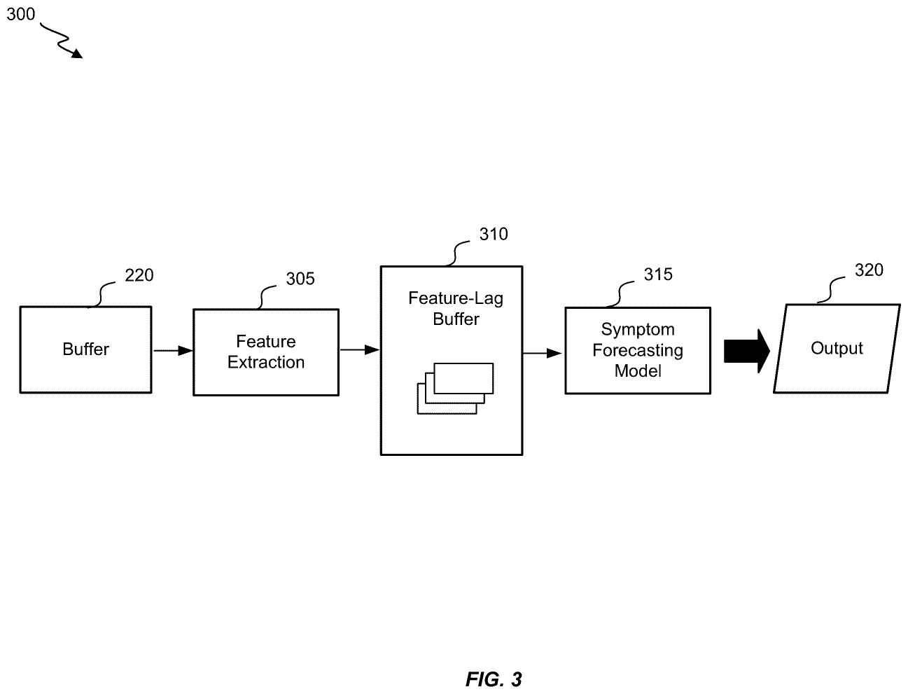

FIG. 3 illustrates an exemplary pipeline to process buffered data or multi-modal data for a symptom-forecasting model 315 that predicts the one or more symptom intensity scores. According to the disclosed technique, a feature extraction 305 may extract one or more features from the sampled information and/or data points (which can include symptom-intensity scores) stored in the buffer 220. For instance, the one or more features extracted from the sampled information and/or data points (or the multi-modal data) may correspond to part or all the information and/or data points obtained during a specific time frame (e.g., for every 30 minutes of the day). During the specific time frame, the one or more features extracted from the information and/or data points can represent a severity or intensity of the one or more symptoms.

For example, the one or more features extracted from the sampled information and/or data points may correspond to a percentage of time during which tremors, dyskinesia, or other symptoms were experienced within every 30 minutes of the day. Similarly, the one or more features may be based on a comparison of the average percentage of tremors and/or dyskinesia with a value of the percentage of tremors and/or dyskinesia usually experienced by the subject (e.g., historical data pertaining to the percentage of tremors and/or dyskinesia experienced by the subject). In addition, the one or more features can be based on a comparison of the average percentage of tremors and/or dyskinesia with a standard value of the percentage of tremors and/or dyskinesia usually experienced by one or more subjects with similar severity of the neurodegenerative disorder, and the like.

In some instances, the one or more features extracted by the feature extraction 305 may further includes predicting an attribution of the average percentage of tremors and/or dyskinesia with the medication information in the specific time frame. For instance, attributing the average percentage of tremors and/or dyskinesia with the medication information in the specific time frame includes sampling the percentage of intensity of the one or more symptoms (e.g., tremors, slowness of movement, dyskinesia, and the like) in accordance with at least a type of the treatment and/or corrective therapy, a dosage of the medicine prescribed in the treatment, a time of the treatment and/or corrective therapy, and the like.

Further features can be defined based on the information calculated and/or collected for each time period of the one or more time periods in the specific time frame. For example, time derivatives can be computed over preceding and successive time periods. Thus, a variety of features may be calculated based on the multi-modal data of the subject. Some features may correspond to individual time period, while others may apply to multiple or all time periods associated with the specific time frame (e.g., last 24 hours, last week, etc.)

In some instances, a feature-lag buffer 310 is configured to store the one or more features extracted using the feature extraction 305. In some instances, the feature-lag buffer 310 may be a temporary or a permanent storage location associated with the intelligent symptom monitoring, forecasting, and management system. The feature-lag buffer 310 may include but is not limited to data storage devices such as computer memory of the computing device 110 or the user device (e.g., smartwatch, smartphone, tablet etc.), or computer memory of the cloud server 115 and the like.

According to some aspects of the present disclosure, the symptom-forecasting model 315 may be utilized to predict a symptom-intensity score for each of the one or more symptoms based on the extracted one or more features in the specific time frame. For instance, a data processor associated with the intelligent symptom monitoring and forecasting system determines that the feature-lag buffer 310 is “full”, or it is determined that a status associated with the capacity of the feature-lag buffer 310 is flagged as “complete” in the specific time frame. In some instances, once it is determined that the feature-lag buffer 310 is “full” or the capacity of the feature-lag buffer 310 is flagged as “complete,” the one or more features stored in the feature-lag buffer 310 is integrated with the samples (or current samples) of the information and/or data points stored in the buffer 220.

In some instances, the transmitter may transmit the integrated features that include the one or more features stored in the feature-lag buffer 310 and the samples of the information and/or data points stored in the buffer 220, to the computing device 110 associated with the intelligent symptom monitoring, forecasting, and management system. The computing device 110 may facilitate the use of the symptom-forecasting model 315 to generate the predicted symptom-intensity score based on the integration of the one or more features that are stored in the feature-lag buffer 310 and the samples of the information and/or data points in the buffer 220. The symptom-forecasting model 315 can include a machine-learning model, such as a generative model, a transformer model, a bidirectional encoder representations from transformers (BERT) model, a Word2Vec model, multi-value prediction algorithm or auto-regressive algorithm.

In some embodiments, the symptom-forecasting model 315 may be trained on the labeled dataset of multiple subjects on the cloud server 115. Moreover, the symptom-forecasting model 315 may be deployed on the cloud server 115 as a cloud service. In some other embodiments, the symptom-forecasting model 315 may be fine-tuned or re-trained using the data of the subject to achieve personalization and better accuracy. The symptom-forecasting model 315 can be deployed on the computing device 110 as edge AI.

The symptom-forecasting model 315 may generate an output 320 based on the one or more features alone or in some instances, based on the integration of the one or more features with the samples of the information and/or data points. The output 320 of the symptom-forecasting model 315 may include the predicted symptom-intensity score corresponding to each symptom among the one or more symptoms in one or more future time periods. The predicted symptom-intensity score indicates a predicted severity or a degree of symptom (e.g., tremors, dyskinesia, or dystonia etc.) that the subject will experience during a future time period. The predicted symptom-intensity score may include, for example, a number (e.g., an integer or real number selected from a defined scale) or a category. The categories can be mild, moderate, or high severity. In the case of relative assessment as compared to symptom-intensity score of current time period, the categories may be based on relative terms such as increase/up, decrease/down, or same/stable and the like. The predicted symptom-intensity score can represent an absolute symptom intensity or a relative symptom intensity (e.g., relative to a symptom intensity associated with a current and/or recent time period). In some instances, the predicted symptom-intensity score may further be based on the computation of average percentage of tremors and/or dyskinesia in a future time period, comparison of the average percentage of tremors and/or dyskinesia with a value of the percentage of tremors and/or dyskinesia usually experienced by the subject (for e.g., historical data pertaining to the percentage of tremors and/or dyskinesia experienced by the subject), comparison of the average percentage of tremors and/or dyskinesia with a standard value of the percentage of tremors and/or dyskinesia usually experienced by one or more subjects with similar severity of the neurodegenerative disorder, and the like.

In some instances, a trend associated with the one or more symptoms is generated based on the symptom-intensity score(s) and/or predicted symptom-intensity score(s). For instance, the computing device 110 may be configured to use the symptom-forecasting model 315 to generate the trend, which can predict how the symptom intensity will vary with time for the subject. The trend may include, for each of one or more future data points, a predicted symptom-intensity score. The symptom-forecasting model 315 may generate the trend by using symptom-intensity scores associated with a current and/or one or more past time periods (e.g., to fit a function, learn one or more parameters, etc.). The symptom-forecasting model 315 may include techniques but are not limited to linear regression, polynomial regression, exponential smoothing, moving averages (e.g., autoregressive integrated moving average), non-parametric methods (e.g., locally estimated scatterplot smoothing), for identifying the underlying pattern or relationship that connects the points (symptom-intensity scores) over time. In some instances, the trend may include only one predicted symptom-intensity score corresponding to only a single future time period. Such a trend may nonetheless be generated based on symptom-intensity scores associated with a current time period and/or one or more past time periods.

The predicted symptom-intensity score(s) may be used to generate the output 320 with the predicted symptom-intensity score(s) and/or with one or more recommendations to improve the quality of life of the subject. The generated one or more recommendations may include a recommendation to: maintain a current schedule for taking a medication, change a schedule for taking the medication, take a dosage of the medication in the near future, engage in one or more forms of exercise (e.g., which may be specified or suggested), etc. A recommendation may be identified using (for example) a look-up table, a decision-tree, a regression model (e.g., that relates a trend or predicted severity score to a variable in the recommendation or urgency of the recommendation, etc.).

In some instances, the output 320 comprising of one or more predicted symptom-intensity scores, a predicted trend, a recommendation, etc. may be transmitted to another computing device (e.g., a smart phone associated with the subject, a computer associated with the subject, a device of a medical provider that is facilitating treatment of the subject, etc.) directly or via the cloud server 115. In some instances, the output 320 can include updating a user interface or a graphical user interface (GUI) of the mobile-app and/or the cloud-app to display the output data. The GUI may be configured to include one or more input components to receive user input that specifies details about how to update the predictions of the symptom-forecasting model 315, to accept/reject a recommendation, and/or to trigger another communication or data-storage action. For example, the one or more input components can be configured to receive input that identifies: (i) sensitivity of symptom changes (e.g., +/−5 minutes, +/−10 minutes) used in the decision process to identify a trend, (ii) the future time periods for predictions (e.g. 10, 30 or 60 minutes), and/or (iii) the symptom being predicted (e.g. tremor, dyskinesia). As another example, the one or more input components can be configured to receive input that indicates whether the subject or the user accepts a recommendation (e.g., thereby indicating that the recommended action was performed or will be performed). As yet another example, the one or more input components can be configured to receive input that indicates whether a user (e.g., a medical-care provider) agrees with or rejects a recommendation, which may trigger a communication indicating such a response to a device of the subject. According to present disclosure, the detection of delayed ‘ON’ state and/or ‘OFF’ state may also be determined in real-time or near real-time and consequently an alert may be generated for the subject and/or the caregiver for timely interventive actions.

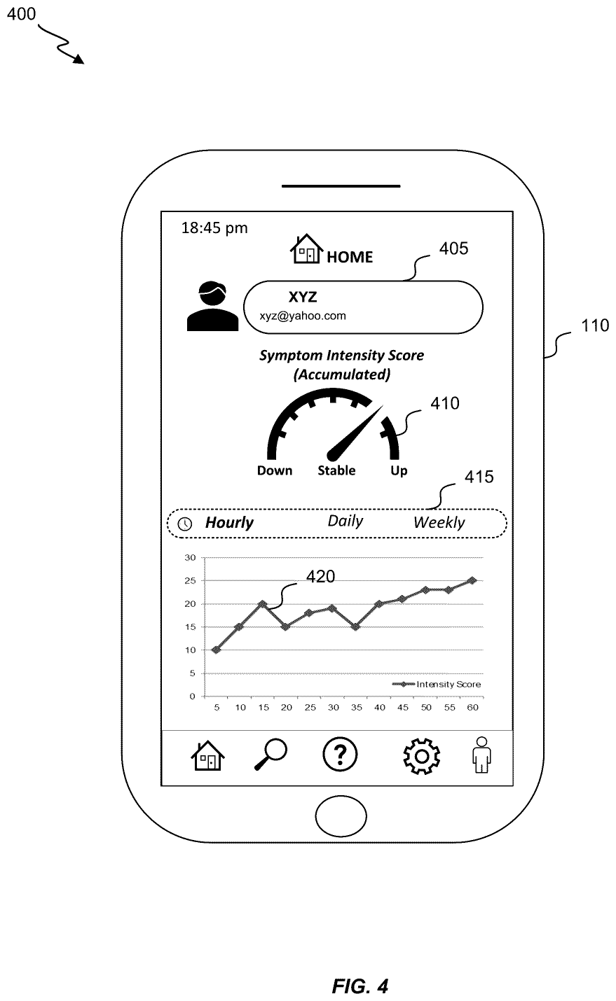

FIG. 4 shows an illustrative example of a first interface 400 of the user interface of the application that can run on the computing device 110. The first interface 400 includes an intensity meter 410 followed by an intensity trend 420. At section 405 of the first interface 400, details or personal details of the subject can be displayed which may include a subject name, an email address, and a profile picture. This is followed by the intensity meter 410 which may display an accumulated or average symptom-intensity score based on the predicted symptom-intensity scores that are generated by the symptom-forecasting model 315. The intensity meter 410 can have values on a scale of ‘0’ to ‘10’ to represent the absolute symptom-intensity score. A symptom-intensity score of value ‘0’ may indicate no symptoms and a value of ‘10’ can be indicative of very severe symptoms or dangerous state. In some instances, the average symptom-intensity score based on the predicted symptom-intensity scores of future time periods can be compared with the current symptom-intensity score of the subject. Based on the comparison, the intensity meter 410 may indicate a relative status of symptoms for the future time period such as ‘Down’, ‘Stable’, and ‘Up’. The relative status ‘Down’ may indicate that the symptom-intensity will decrease in future time period, ‘Stable’ indicates a steady or similar value as compared to current or near past time periods, and ‘Up’ may represent a rise or increase in symptoms intensity.

In the bottom of the illustrative example of the first interface 400, an intensity trend 420 can be plotted with the symptom-intensity score on the y-axis and a selected time frame on the x-axis. In some instances, the intensity trend 420 may include few initial data points (or symptom-intensity scores) corresponding to near past and/or current time periods and other data points may represent predicted symptom-intensity scores of a selected symptom. In some other instances, the intensity trend 420 may only include predicted symptom-intensity scores of the selected symptom for future time periods. In some other instances, the intensity trend 420 may only be based on the symptom-intensity scores associated with the past time periods that are saved in the database 120 or in the local database of the computing device 110 for visualization of the subject or the clinician. For example, in the depicted instance of the intensity trend 420, the selected time frame is hourly, and the symptom-intensity score is plotted for a time interval of 5 mins. A time-selection bar 415 may allow the subject to select a time frame for a visualization of the trend such as on an hourly, daily or weekly basis. Based on the selection of the subject, the time interval can also vary. For example, if the user selects weekly, the time interval can be one day between the data points of the plot for a weekly trend.

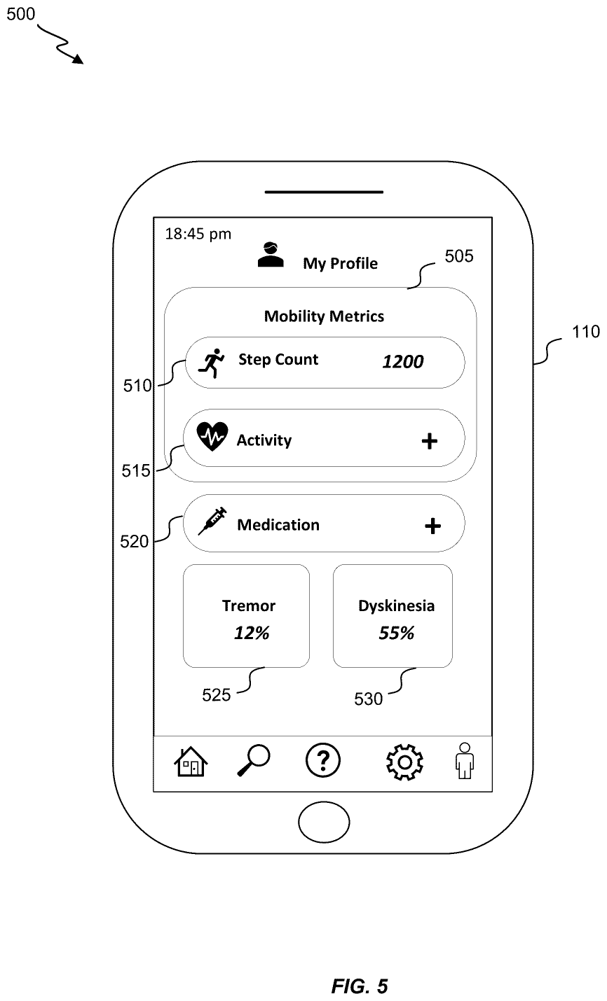

FIG. 5 shows an illustrative example of a second interface 500 of the user interface of the application that can run on the computing device 110 or the user device. The second interface 500 may include mobility metrics 505, medication 520, percentage tremor 525 and percentage dyskinesia 530. The mobility metrics 505 may refer to physical movements or activity type and level of the subject. The mobility metrics 505 can be used to track a physical activity progress over time for the subject. The mobility metrics 505 may further include a step count 510 and an activity 515, which can either be automatically monitored based on the sensor data 205 or manually updated by the subject on the second interface 500 of the application. The activity 515 may include resting, walking, running, jumping, cycling, stretching and the like. Keeping track of the mobility metrics 505 can facilitate maintaining or improving mobility while aiding in controlled motor movements.

In some instances, the medication 520 may correspond to the medications data (e.g., medicines prescribed, dosage etc.) retrieved from the database 120 or the medications that are due or already taken by the subject. In some other instances, the medicines to be taken by the subject may be listed in the medication 520 and the subject can simply check the box after taking the medicines. The subject or user provided medication information and the mobility metrics 505 of recent time periods can improve the accuracy of the predicted symptom-intensity score. A notification mechanism can also be added in the application to alert the subject for timely medication. Additionally, the second interface 500 may also display the one or more symptom intensity scores, for example, percentage tremor 525 and percentage dyskinesia 530 which are derived based on the sensor data of the current time period by using the symptom-intensity model 210. A threshold may be applied on each of the percentage tremor 525 and percentage dyskinesia 530 values and may trigger an alert for higher values.

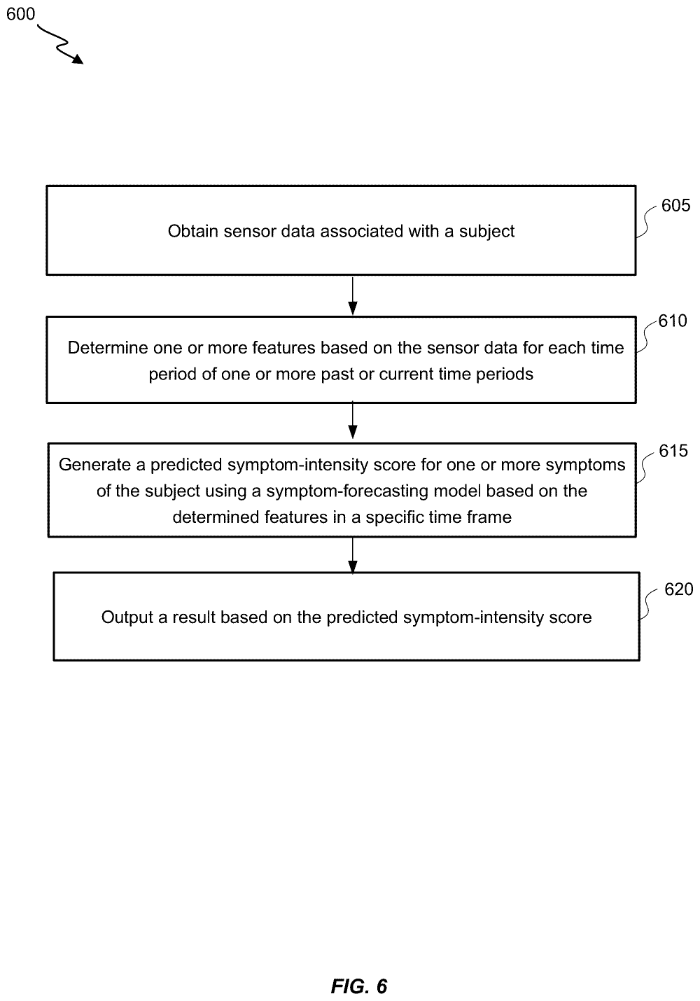

FIG. 6 shows an example flowchart of a system to predict the one or more symptom-intensity scores of the subject in accordance with some embodiments of the present disclosure. The blocks in flowchart 600 are illustrated in a specific order, while the order can be modified, for example, some blocks may be performed before other, and some blocks may be performed simultaneously. The blocks can be performed by hardware or software or a combination thereof. The process at block 605 may include obtaining the sensor data 205 of the subject for one or more past or current time periods. The sensor data 205 may include but not limited to accelerometer signals, gyroscope signals, EMG signals. The symptom-intensity model 210 may utilize the sensor data 205 to generate a symptom-intensity score for each of the one or more symptoms of the subject and for each time period of the one or more past or current time periods.

One or more features may be extracted based on the sensor data 205 for each time period of the one or more past or current time periods, at block 610. The one or more features that are extracted for each time period may be determined using the symptom-intensity score. A predicted symptom-intensity score for one or more symptoms of the subject may be generated based on the one or more features that are extracted in a specific time frame and by using the symptom-forecasting model 315, at block 615. The specific time frame may include current time period and one or more past time periods. In some instances, the predicted symptom-intensity score corresponds to the predicted intensity of the one or more symptoms of Parkinson's disease. In some other instances, a trend may be generated using the symptom-intensity scores corresponding to the one or more past or current time periods. Afterwards, the trend can be used to generate the predicted symptom-intensity score. In some other embodiments, the predicted symptom-intensity score may be generated based on multi-modal data comprising the sensor data 205, the medical information, and mobility metrics of the subject. The mobility metrics of the subject can be derived from the sensor data 205.

A result may be output or generated based on the predicted symptom-intensity score, at block 620. The result may provide a basis for a recommendation or includes the recommendation to perform an intervening action to avoid symptoms in accordance with the predicted symptom-intensity score. The result or output may include an alert that the one or more symptoms of the neurodegenerative disorder may get worse in the future time period without the intervening action. The result, the trend, and/or the alert may be presented on a device (e.g., smartphone, tablet, laptop etc.) of the subject, caregiver, or a clinician or may be transmitted to another device, remote device, or a cloud server.

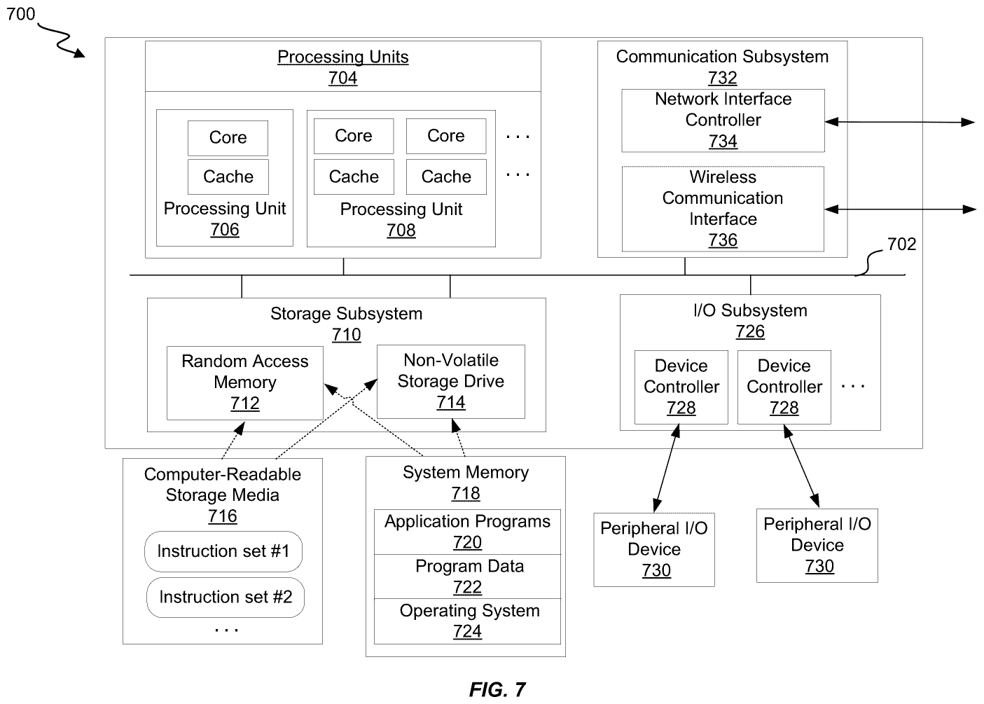

FIG. 7 shows an example illustration of a computer system 700 in which various embodiments of the present disclosure may be implemented. The computer system 700 can be used as the computing device 110 as explained in FIG. 1. For example, the techniques as disclosed above in the present disclosure for monitoring, forecasting, and management of the unintended motor movements using multi-modal data can be implemented in computer-executable instructions (e.g., organized in application programs 720). The application programs 720 can include the routines, programs, objects, components, and data structures that perform the tasks and implement the data types for implementing the techniques described above. The functionality described herein can be performed, at least in part, by one or more hardware logic components.

In the exemplary block diagram, the computer system 700 includes processing units 704 that communicate with several peripheral subsystems via a bus subsystem 702. These peripheral subsystems include, for example, a storage subsystem 710, an I/O subsystem 726, and a communication subsystem 732. The bus subsystem 702 provides a mechanism for letting the various components and subsystems of the computer system 700 to communicate with each other as intended. Although the bus subsystem 702 is shown schematically as a single bus, alternative embodiments of the bus subsystem may utilize multiple buses. Bus subsystem 702 may be any of several types of bus structures including a memory bus or memory controller, a peripheral bus, and a local bus using any of a variety of bus architectures. Such architectures may include, for example, an industry standard architecture (ISA) bus, micro channel architecture (MCA) bus, enhanced ISA (EISA) bus, video electronics standards association (VESA) local bus, and peripheral component interconnect (PCI) bus, which can be implemented as a mezzanine bus manufactured to the IEEE P1386.1 standard.

The processing units 704, which may be implemented as one or more integrated circuits (e.g., a microprocessor or microcontroller), control the operation of the computer system 700. One or more processors, including single core and/or multicore processors, may be included in the processing units 704. As shown in FIG. 7, the processing units 704 may be implemented as one or more independent processing units 706 and/or 708 with single or multicore processors and processor caches included in each processing unit. In some embodiments, the processing units 704 may also be implemented as a quad-core processing unit or larger multicore designs (e.g., hexa-core processors, octa-core processors, ten-core processors, or greater. In some cases, the processing units 704 may include one or more specialized ASICs designed and configured for cryptocurrency mining and/or specialized cryptographic hardware for handling cryptocurrency transactions.

The processing units 704 may execute a variety of software processes embodied in a program code and may maintain multiple concurrently executing programs or processes. At any given time, some or all the program code to be executed can be resident in the processing units 704 and/or in the storage subsystem 710. In some embodiments, the computer system 700 may further include one or more specialized processors, such as digital signal processors (DSPs), outboard processors, graphics processors, application-specific processors, and/or the like.

The I/O subsystem 726 may include device controllers 728 for one or more user interface devices such as peripheral I/O devices 730. The peripheral I/O devices 730 may be integral with the computer system 700 (e.g., integrated audio/video systems, and/or touchscreen displays), or may be separate that can be attachable/detachable from the computer system 700, one or more input devices may include keyboard, mouse, pen, voice input device, touch input device, etc. Similarly, one or more output devices may include displays, speakers, printers, etc. These devices are well known in the art and are not discussed at length here.

The computer system 700 may comprise one or more storage subsystems 710, comprising hardware and software components used for storing data and program instructions, such as a computer-readable storage media 716 and a system memory 718. The system memory 718 and/or the computer-readable storage media 716 may store program instructions that are loadable and executable on the processing units 704, as well as data generated during the execution of these programs. Depending on the configuration and type of the computer system 700, the system memory 718 may be stored in a volatile memory, such as random-access memory (RAM) 712, and/or in a non-volatile storage drive 714 (e.g., read-only memory (ROM), flash memory etc.).

By way of example, and not limitation, the system memory 718 also may also include application programs 720, which may include client applications, Web browsers, mid-tier applications, relational database management systems (RDBMS), etc., program data 722, and an operating system 724. By way of example, operating system 724 may include various versions of Microsoft Windows®, Apple Macintosh®, and/or Linux operating systems, a variety of commercially-available UNIX® or UNIX-like operating systems (including without limitation the variety of GNU/Linux operating systems, the Google Chrome OS, and the like) and/or mobile operating systems such as iOS, Windows® Phone, Android OS, BlackBerry® OS, and Palm® OS operating systems. All or portions of operating system 724, application programs 720, and/or program data 722 can also be cached in memory such as the volatile memory and/or non-volatile memory, for example (RAM 712 or non-volatile storage drive 714 such as ROM). It is to be appreciated that the disclosed architecture can be implemented with various commercially available operating systems or combinations of operating systems (e.g., virtual machines).

The storage subsystem 710 may also provide one or more tangible computer-readable storage media 716 for storing the basic programming and data constructs that provide the functionality of some embodiments. Software (programs, code modules, instructions) that when executed by a processor provide the functionality described herein may be stored in the storage subsystem 710. These software modules or instructions may be executed by the processing units 704. The storage subsystem 710 may also provide a repository for storing data used in accordance with the present disclosure. The storage subsystem 710 may also include a computer-readable storage media reader that can further be connected to computer-readable storage media 716. Together and, optionally, in combination with the system memory 718, the computer-readable storage media 716 may comprehensively represent remote, local, fixed, and/or removable storage devices plus storage media for temporarily and/or more permanently containing, storing, transmitting, and retrieving computer-readable information.

Computer-readable storage media 716 containing program code, or portions of program code, may include any appropriate media known or used in the art, including storage media and communication media, such as but not limited to, volatile and non-volatile, removable and nonremovable media implemented in any method or technology for storage and/or transmission of information. This can include tangible computer-readable storage media such as RAM, ROM, electronically erasable programmable ROM (EEPROM), flash memory or other memory technology, CD-ROM, digital versatile disk (DVD), or other optical storage, magnetic cassettes, magnetic tape, magnetic disk storage or other magnetic storage devices, or other tangible computer readable media. This can also include nontangible computer-readable media, such as data signals, data transmissions, or any other medium which can be used to transmit the desired information, and which can be accessed by the computer system 700.

By way of example, computer-readable storage media 716 may include a hard disk drive that reads from or writes to non-removable, nonvolatile magnetic media, a magnetic disk drive that reads from or writes to a removable, nonvolatile magnetic disk, and an optical disk drive that reads from or writes to a removable, nonvolatile optical disk such as a CD-ROM, DVD, and Blu-Ray® disk, or other optical media. Computer-readable storage media 716 may include, but is not limited to, Zip® drives, flash memory cards, universal serial bus (USB) flash drives, secure digital (SD) cards, DVD disks, digital video tape, and the like. Computer-readable storage media 716 may also include, solid-state drives (SSD) based on non-volatile memory such as flash-memory based SSDs, enterprise flash drives, solid state ROM, and the like, SSDs based on volatile memory such as solid state RAM, dynamic RAM, static RAM, DRAM-based SSDs, magneto resistive RAM (MRAM) SSDs, and hybrid SSDs that use a combination of DRAM and flash memory based SSDs. The disk drives and their associated computer readable media may provide non-volatile storage of computer-readable instructions, data structures, program modules, and other data for the computer system 700.

A communication subsystem 732 may provide a communication interface from the computer system 700 and external computing devices via one or more communication networks, including local area networks (LANs), wide area networks (WANs) (e.g., the internet), and various wireless telecommunications networks. The communication subsystem 732 may include, for example, one or more network interface controllers (NICs) 734, such as ethernet cards, asynchronous transfer mode NICs, token ring NICs, and the like, as well as one or more wireless communication interfaces 736, such as wireless network adapters, and the like. Additionally and/or alternatively, the communication subsystem 732 may include one or more modems (telephone, satellite, cable, ISDN), synchronous or asynchronous digital subscriber line (DSL) units, and the like. The communication subsystem 732 also may include radio frequency (RF) transceiver components for accessing wireless voice and/or data networks (e.g., 4G, 5G, etc.), Wi-Fi, global positioning system (GPS) receiver components. The various physical components of the communication subsystem 732 may be detachable components coupled to the computer system 700 via a computer network, and/or may be physically integrated onto a motherboard of the computer system 700. The communication subsystem 732 also may be implemented in whole or in part by software.

In some embodiments, the communication subsystem 732 may also receive input communication in the form of structured and/or unstructured data feeds, event streams, event updates, and the like, on behalf of one or more users who may use or access the computer system 700. For example, the communication subsystem 732 may be configured to receive data feeds in real-time from users of social networks and/or other communication services, web feeds such as rich site summary (RSS) feeds, and/or real-time updates from one or more third party information sources. Additionally, the communication subsystem 732 may be configured to receive data in the form of continuous data streams, which may include event streams of real-time events and/or event updates (e.g., sensor data applications, alerts, notifications, financial tickers, network performance measuring tools, clickstream analysis tools, etc.). Communication subsystem 732 may output such structured and/or unstructured data feeds, event streams, event updates, and the like to one or more data stores that may be in communication with one or more streaming data source computers coupled to the computer system 700.

In some aspects, the computer system 700 can be one of several computers employed in a datacenter and/or computing resources (hardware and/or software) in support of cloud computing services for portable and/or mobile computing systems such as wireless communications devices, cellular telephones, and other mobile-capable devices. Cloud computing services, include, but are not limited to, infrastructure as a service, platform as a service, software as a service, storage as a service, desktop as a service, data as a service, security as a service and APIs (application program interlaces) as a service, for example.

Other well-known computer systems, environments and/or configurations that may be suitable for use with the embodiments include, but are not limited to personal computers, server computers, hand-held or laptop devices, multiprocessor systems, microprocessor-based systems, set top boxes, game con soles, programmable consumer electronics, network PCs, minicomputers, mainframe computers, distributed computing environments that include any of the above systems or devices, and/or the like. For example, some or all of the components of computer system 700 may be implemented in a cloud computing environment, such that resources and/or services are made available via a computer network for selective use by the user devices.

Due to the ever-changing nature of computers and networks, the description of the computer system 700 depicted in FIG. 7 is intended only as a specific example. Many other configurations having more or fewer components than the system depicted in FIG. 7 are possible. For example, customized hardware might also be used and/or particular elements might be implemented in hardware, firmware, software, or a combination. Further, connection to other computing devices, such as network input/output devices, may be employed. Based on the disclosure and teachings provided herein, a person of ordinary skill in the art will appreciate other ways and/or methods to implement the various embodiments.

Some embodiments of the present disclosure include a system including one or more data processors. In some embodiments, the system includes a non-transitory computer readable storage medium containing instructions which, when executed on the one or more data processors, cause the one or more data processors to perform part or all of one or more methods and/or part or all of one or more processes disclosed herein. Some embodiments of the present disclosure include a computer-program product tangibly embodied in a non-transitory machine-readable storage medium, including instructions configured to cause one or more data processors to perform part or all of one or more methods and/or part or all of one or more processes disclosed herein.

The terms and expressions which have been employed are used as terms of description and not of limitation, and there is no intention in the use of such terms and expressions of excluding any equivalents of the features shown and described or portions thereof, but it is recognized that various modifications are possible within the scope of the invention claimed. Thus, it should be understood that although the present invention as claimed has been specifically disclosed by embodiments and optional features, modification, and variation of the concepts herein disclosed may be resorted to by those skilled in the art, and that such modifications and variations are considered to be within the scope of this invention as defined by the appended claims.

The present description provides preferred exemplary embodiments only, and is not intended to limit the scope, applicability or configuration of the disclosure. Rather, the description of the preferred exemplary embodiments will provide those skilled in the art with an enabling description for implementing various embodiments. It is understood that various changes may be made in the function and arrangement of elements without departing from the spirit and scope as set forth in the appended claims.

Specific details are given in the following description to provide a thorough understanding of the embodiments. However, it will be understood that the embodiments may be practiced without these specific details. For example, circuits, systems, networks, processes, and other components may be shown as components in block diagram form in order not to obscure the embodiments in unnecessary detail. In other instances, well-known circuits, processes, algorithms, structures, and techniques may be shown without unnecessary detail in order to avoid obscuring the embodiments.

What is claimed is:

1. A computer-implemented method comprising:
obtaining sensor data associated with a subject;
extracting, for each time period of one or more past or current time periods, one or more features based on the sensor data;
generating, using the extracted one or more features in a specific time frame, a predicted symptom-intensity score for one or more symptoms of the subject using a symptom-forecasting model, wherein the predicted symptom-intensity score represents a predicted intensity of the one or more symptoms during a future time period; and
outputting a result based on the predicted symptom-intensity score, wherein the result provides a basis for a recommendation or includes the recommendation to perform an intervening action to avoid symptoms in accordance with the predicted symptom-intensity score.

2. The computer-implemented method of claim 1, further comprising:
determining, for each time period of the one or more past or current time periods, a symptom-intensity score based on at least a portion of the sensor data that corresponds to the time period, wherein the one or more features for the time period are determined using the symptom-intensity score.

3. The computer-implemented method of claim 1, wherein the predicted symptom-intensity score corresponds to a predicted intensity of the one or more symptoms of Parkinson's disease.

4. The computer-implemented method of claim 1, wherein the predicted symptom-intensity score corresponds to a predicted intensity of tremors or a predicted intensity of dyskinesia.

5. The computer-implemented method of claim 1, wherein the sensor data comprises data collected by an accelerometer or a gyroscope.

6. The computer-implemented method of claim 1, wherein the predicted symptom-intensity score is further generated based on medical information comprising dosage information associated with a treatment of the subject, medication time delta information, and information regarding effect of the treatment on the subject.

7. The computer-implemented method of claim 1, wherein generating the predicted symptom-intensity score includes generating a trend using symptom-intensity scores corresponding to the one or more past or current time periods, and wherein the predicted symptom-intensity score is based on the trend.

8. The computer-implemented method of claim 1, wherein the predicted symptom-intensity score is further generated based on multi-modal data comprising the sensor data, medical information, and mobility metrics of the subject.

9. A system comprising:
one or more data processors; and
a non-transitory computer readable storage medium containing instructions which, when executed on the one or more data processors, cause the one or more data processors to perform a set of operations including:
obtaining sensor data associated with a subject;
extracting, for each time period of one or more past or current time periods, one or more features based on the sensor data;
generating, using the extracted one or more features in a specific time frame, a predicted symptom-intensity score for one or more symptoms of the subject using a symptom-forecasting model, wherein the predicted symptom-intensity score represents a predicted intensity of the one or more symptoms during a future time period; and
outputting a result based on the predicted symptom-intensity score, wherein the result provides a basis for a recommendation or includes the recommendation to perform an intervening action to avoid symptoms in accordance with the predicted symptom-intensity score.

10. The system of claim 9, wherein the predicted symptom-intensity score corresponds to a predicted intensity of the one or more symptoms of Parkinson's disease.

11. The system of claim 9, wherein the predicted symptom-intensity score corresponds to a predicted intensity of tremors or a predicted intensity of dyskinesia.

12. The system of claim 9, wherein the sensor data comprises data collected by an accelerometer or a gyroscope.

13. The system of claim 9, wherein the predicted symptom-intensity score is further generated based on medical information comprising dosage information associated with a treatment of the subject, medication time delta information, and information regarding effect of the treatment on the subject.

14. The system of claim 9, wherein generating the predicted symptom-intensity score includes generating a trend using symptom-intensity scores corresponding to the one or more past or current time periods, and wherein the predicted symptom-intensity score is based on the trend.

15. A computer-program product tangibly embodied in a non-transitory machine-readable storage medium, including instructions configured to cause one or more data processors to perform a set of operations comprising:
obtaining sensor data associated with a subject;
extracting, for each time period of one or more past or current time periods, one or more features based on the sensor data;
generating, using the extracted one or more features in a specific time frame, a predicted symptom-intensity score for one or more symptoms of the subject using a symptom-forecasting model, wherein the predicted symptom-intensity score represents a predicted intensity of the one or more symptoms during a future time period; and
outputting a result based on the predicted symptom-intensity score, wherein the result provides a basis for a recommendation or includes the recommendation to perform an intervening action to avoid symptoms in accordance with the predicted symptom-intensity score.

16. The computer-program product of claim 15, further comprising:
determining, for each time period of the one or more past or current time periods, a symptom-intensity score based on at least a portion of the sensor data that corresponds to the time period, wherein the one or more features for the time period are determined using the symptom-intensity score.

17. The computer-program product of claim 15, wherein the predicted symptom-intensity score corresponds to a predicted intensity of the one or more symptoms of Parkinson's disease.

18. The computer-program product of claim 15, wherein the predicted symptom-intensity score is further generated based on medical information comprising dosage information associated with a treatment of the subject, medication time delta information, and information regarding effect of the treatment on the subject.

19. The computer-program product of claim 15, wherein generating the predicted symptom-intensity score includes generating a trend using symptom-intensity scores corresponding to the one or more past or current time periods, and wherein the predicted symptom-intensity score is based on the trend.

20. The computer-program product of claim 15, wherein the predicted symptom-intensity score is further generated based on multi-modal data comprising the sensor data, medical information, and mobility metrics of the subject.
